(19) United States
(12) Patent Application Publication
(10) Pub. No.: US 2025/0191711 A1
(43) Pub. Date: Jun. 12, 2025

(54) MEDICAL DEVICE SYSTEM FOR PATIENT HEALTH CARE INFORMATION COLLECTION, COMMUNICATION AND MANAGEMENT

(71) Applicant: MedStreamline LLC, Greenville, SC (US)

(72) Inventor: Mohamed Hassan Behery, Greenville, SC (US)

(73) Assignee: MedStreamline LLC, Greenville, SC (US)

(21) Appl. No.: 19/054,868

(22) Filed: Feb. 16, 2025

Related U.S. Application Data
(63) Continuation of application No. 17/881,709, filed on Aug. 5, 2022, now abandoned.
(60) Provisional application No. 63/229,116, filed on Aug. 4, 2021.

(51) Int. Cl. G16H 10/60; G16H 40/67

(52) U.S. Cl. CPC G16H 10/60; G16H 40/67

(57) ABSTRACT

A system for data capture and storage comprising: a data capture device taken from the group consisting of blood pressure device, thermometer, pulse sensor, scale and glucose meter; a local computer system in communication with the data capture device; a set of local computer readable instructions configured for receiving data from the data capture device, display the data capture device in a user-friendly format, determine if there is an electronic communications link and if so, transmit the data to a remote computer system, retrieve data from the remote computer system and display a combination of the data frohe data capture device and the remote computer system to the user.

### RELATED APPLICATIONS

This application is a continuation application from U.S. patent application Ser. No. 17/881,709 filed Aug. 5, 2022 which claims priority from United States Provisional Patent Application 63/229,116 filed Aug. 4, 2021 each of which is incorporate by reference.

### BACKGROUND OF THE INVENTION

### 1) Field of the Invention

This system is directed to the collection and storage of medical information, especially triage information, offering a distributed collection and distributed storage that can be integrated, uploaded and/or synchronized with an electronic medical records system.

### 2) Description of the Related Art

Medical providers rely upon a large amount of information that is collected from a patient for practical and regulatory reasons. Under one study, health care providers fill out an average of 20,000 forms each year. One study found that for every hour physicians provide direct clinical face time to patients, nearly 2 additional hours are spent on electronic medical records (EMR) and desk work within the clinic day. This study found that physicians spent 52.9% of their time on direct clinical face time and 37.0% on EMR and desk work. Further, some physicians reported that they spend 1 to 2 hours of after-hours work each night, devoted mostly to EMR tasks. It has been reported that 38 percent of healthcare costs go to administrative overhead.

Therefore, there is a need to improve the medical data recording process so that less time is spent on administrative tasks, more time is spent on providing health care services, and the overall efficiency of the health care process, as well as the industry, can be improved.

Further, when health care data is recorded, there is the risk of error in the collection and recording of the data. One source of error is in the manual process that is used in most settings. This is a multi-step process for each vital sign taken without any connection or correlation between the multiple steps and vital sign recordings. For example, when taking a first vital sign, the health care provider uses a device (e.g., sensor) to measure health information (e.g., blood pressure reader), administers the device, reads the information from the medical device, and enters the results in an EMR system. Further, the consistency of the readings are not monitors and the current system simply relies upon the health care provider to take consistent readings and to use the medical device correctly.

The process of data collection to data recording can result in errors in the EMR information due to transcription errors, information reading errors, improper device usage, and the like. Further, vital signs and other information are taken independently of each other without the ability of the health care provider to associate the data with any series, time, or sequence. The timing, sequency, frequency, and other circumstances can have a profound impact on healthcare and preventable morbidity. One study states that there is great importance of properly taken vital signs as an aid in the identification of deterioration in patients, to identify the need for intensive care unit transfers, and as a component in the emergency room triage. The more the vital signs deviate from the normal range, the larger are the odds of mortality. Errors in the vital sign process can result in unnecessary delay between the time a patient needs to see a health care provider and when the patient sees the health care provider. Errors in the process has been reported to be the third leading cause of death in the United States. Therefore, there is a need for accurate information collection and data collection from the source to the EMR.

Further, it has been reported that errors in collecting, recording, and storing medical information cost approximately 20 billion dollars a year. It has also been reported that the leading cause behind medical errors is the miscommunication of data. These miscommunications can result in missing information, delays as test are reordered, additional costs, lost productivity, and ultimately, heath care services.

There are also regulatory requirements concerning the data collection and storage of medical data. Providing an accurate audit trail of data collection and storage can reduce the risks of regulatory non-compliance. For example, the 2009 Health

Information Technology for Economic and Clinical Health act, hospitals and healthcare providers must be able to demonstrate that their electronic health records' systems are capable of certain tasks that constitute “meaningful use.” Under this regulatory scheme, healthcare providers must meet 19 of 24 “meaningful use” objectives that include electronically tracking patients' medications and allergies, sending reminders, sharing lab test results, and producing summaries of a patient's office visit to receive payment.

There is also a movement to have more health care services provided in an outpatient or pre-visit process as this lowers the costs of healthcare and makes the process more efficient. Therefore, having a system that allows for the initial data collection to be performed prior to arriving at a health care provider would be advantageous. Such a system would need to have data verification so that the health care data can be collected by the patient without the need for the health care provider to collect the data, while also providing accurate and timely health care data.

There have been some attempts to monitor health information “at home” such as shown in U.S. Pat. No. 9,107,586, however, such systems are not integrated with data capture devices, EMR systems, stored in a decentralized manner allowing for multiple secured access, nor do they provide a systematic method for collecting vital signs under dynamic conditions.

There is also a need for a system that can assist with telemedicine, including online medical visits, so that the health care provider has access to timely, accurate medical information of the patient without the patient necessarily having to physically travel to the health care provider location. Attempts to assist with telemedicine includes U.S. Pat. No. 10,780,582, but such attempts are limited in that the patient still must travel to a location out of the home. Further, attempts at health monitoring such as United State U.S. Pat. No. 8,323,188 lack the ability to use accurate data capture devices, securely use common networks, and properly and securely integrate into EMR systems.

There is also a need for a system that can provide for real time dynamic frequency for taking and recording health care information (e.g., vital signs) according to the patterns or trends of the information corrected.

### BRIEF SUMMARY OF THE INVENTION

The above objectives are accomplished by providing a system for data capture and storage comprising: a data capture device taken from the group consisting of blood pressure device, thermometer, pulse sensor, scale and glucose meter; a local computer system in communication with the data capture device; a set of local computer readable instructions configured for receiving data from the data capture device, display the data capture device in a user-friendly format, determine if there is an electronic communications link and if so, transmit the data to a remote computer system, retrieve data from the remote computer system and display a combination of the data from the data capture device and the remote computer system to the user.

The local computer readable instructions can be configured to transmit and store the data from the data capture device encrypted. The remote computer system can be an electronic medical records system. The set of local computer readable instructions can be configured to integrate the data captured from the data capture device into a patient record stored by the electronic medical records system. The data capture device can be configured to store the data from the data capture device if an electronic communications link is not present. The local computer system can be configured to store the data from the data capture device if an electronic communications link is not present. The data capture device can be configured to delete the data from the data capture device after the data is transmitted to the remote computer system. The electronic communication link can be wired, wireless, local, wide area, or a global communications network. The local computer readable instructions can be configured to receive notes from the user associated with the data from the data capture device. The local computer readable instructions can be configured to provide the data from the data capture device using a link to a remote database that can be accessed by the user or a second user. The local computer readable instructions can be configured to provide a status message to the user that the data has been transmitted to the remote computer device. The local computer readable instructions can be configured to provide a status message to the user that the data has been integrated into an electronic medical records system.

The system can include a local computer system having a computer readable medium; a first medical device in communications with the local computer system adapted for capturing a first set of medical data from a patient; a second medical device in communication with the local computer system adapted for capturing a second set of medical data from the patient; a remote computer system in communication with the local computer system; a set of local computer readable instructions adapted to: receive a first medical device information from the first medical device, display a first use instruction according to the first medical device information, receive a first patient medical information from the first medical device, display the first patient medical information on the local computer system, display a first warning according to the first patient medical information being outside a first acceptable range, receive a second medical device information from the second medical device, display a second use instruction according to the second medical device information, receive a second patient medical information from the second medical device, display the second patient medical information on the local computer system, display a second warning according to the second patient medical information being outside a second acceptable range, display a correlation information according to a comparison of the first patient medical information and the second patient medical information, and, transmit the first patient medical information and the second patient medical information to a remote computer system.

When the medical device provides information to the computer system, the computer system can determine several inputs and outputs such as medical device dependent user instructions, protocols for receiving information from the medical device, and the like. Therefore, the computer system does not relay upon a single type of medical device or system. When data is captured from the medical device, it can be automatically or otherwise transmitted to third parties that can include health care providers, first responders, family members, automatic health care system (e.g., EMR), and the like.

The computer system can have its own patient record as well as retrieve, amend, and store a patient record from a third party (e.g., EMR at a health care facility). The patient record can be stored on a local and remote database and can be associated with a patient representing historical medical information. The set of local computer readable instructions can be adapted to modify the patient record according to information taken from a group consisting of the first medical device information, first patient medical information, second medical device record information, second patient medical information, date, time, location, and any combination thereof. Therefore, the data received from a first medical device can be associated with, correlated with, or otherwise operatively associated with, the data from a second medical device. For example, blood pressures can be associated with temperature.

The set of local computer readable instructions can be adapted to retrieve the patient record and display a warning if the first medical device information is outside a range. The range can be health care standards such as the stages established by the American College of Cardiology and the American Heart Association. The range can be one set by the health care provider, such as in the case when a patient is known to routinely have low or high blood pressure. The range can be established according to the history of the patient, such as by using historical data with averages, or other calculations, such as mean with standard deviations taken into consideration.

The set of local computer readable instructions can be adapted to display a correlation of information according to a comparison of the first patient medical information and the second patient medical information wherein the first patient medical information affects the second patient medical information.

The computer system and set of local computer readable instructions can be adapted to receive the first patient medical information from the first medical device by manual entry. The computer system can also include internal sensors such as location sensor, temperature sensor, and the like.

The computer system and set of local computer readable instructions can be adapted to request that the patient is prompted to actuate the first medical device to receive a third medical device information from the first medical device. The set of local computer readable instructions can be adapted to request that the patient is prompted to actuate the first medical device to receive a third medical device information from the first medical device according to a comparison of the first patient medical information within the first acceptable range. The set of local computer readable instructions can be adapted to request that the patient is prompted to actuate the second medical device to receive a third medical device information from the second medical device according to a comparison of the first patient medical information within the first acceptable range.

The computer system and set of local computer readable instructions can be adapted to transmit a health message to a third party such as a health care provider, first responder, family member, friend, caretaker, or other third party according to the first patient medical information being outside the first acceptable range. The transmission can be allowed or disallowed through setting on the computer system and can be conditioned upon approval from the patient either through a prior setting or at the time the computer system determines a transmission should be sent.

The system can include a local computer system having a computer readable medium and in communications with an electronic medical record system; an initial patient record stored on a database in communication with the local computer system; a first medical device in communication with the local computer system adapted for capturing a first set of medical data from a patient; a second medical device in communication with the local computer system adapted for capturing a second set of medical data from the patient; a remote computer system in communication with the local computer system; a set of local computer readable instructions adapted to: receive a first medical device information from the first medical device, display a first use instruction according to the first medical device information, receive a first patient medical information from the first medical device, display the first patient medical information on the local computer system, receive a second medical device information from the second medical device, display a second use instruction according to the second medical device information, receive a second patient medical information from the second medical device, display the second patient medical information on the local computer system, and, create an amended patient record according to the initial patient record and information taken from a group consisting of the first medical device information, first patient medical information, the second medical device information, the second patient medical information, date, time, location, and any combination thereof.

The set of local computer readable instructions can be adapted to retrieve the initial patient record from the electronic medical record system, amend the initial patient record and store an amended patient record on the database wherein the database is included in the electronic medical record system. The set of local computer readable instructions can be adapted to transmit the amended patient record to a remote computer system.

The first medical device can be taken from the group consisting of a blood pressure sensor, pulse sensor, thermometer, weight scale, and galvanic skin sensor.

The set of local computer readable instructions can be adapted to instruct the patient to actuate the first medical device after a period of time from receiving the first patient medical information and receiving a third patient medical information from the first medical device.

The system can include a local computer system having a computer readable medium and in communication with an electronic medical record system; an initial patient history stored on a database in communication with the local computer system wherein the initial patient history is associated with a patient; a first medical device in communication with the local computer system adapted for capturing a first set of medical data from the patient; a second medical device in communication with the local computer system adapted for capturing a second set of medical data from the patient; a set of local computer readable instructions adapted to: receive a first medical device information from the first medical device, display a first use instruction according to the first medical device information, receive a first patient medical information from the first medical device, receive a second medical device information from the second medical device, display a second use instruction according to the second medical device information, receive a second patient medical information from the second medical device, append the initial patient history according to information taken from the group consisting of the first patient medical information, the first medical device information, the second patient medical information, the second medical device information, date, time, location, and any combination thereof. The database can be included in the electronic medical records system.

The set of local computer readable instructions can be adapted to append the initial patient history by associating information taken from the group consisting of the first patient medical information, the first medical device information, the second patient medical information, the second medical device information, date, time, location, and any combination thereof, with the initial patient history. The set of local computer readable instructions can be adapted to instruct the patient to actuate the first medical device after a period of time from receiving the first patient medical information and receiving a third patient medical information from the first medical device. The set of local computer readable instructions can be are adapted to transmit a health message to a health care provider according to the first patient medical information being outside a first acceptable range.

### BRIEF DESCRIPTION OF THE SEVERAL VIEWS OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 is schematic of aspects of the system.

FIG. 2 is a flowchart of aspects of the system;

FIG. 3 is a flowchart of aspects of the system;

FIG. 4 is a flowchart of aspects of the system;

FIG. 5 is a flowchart of aspects of the system;

FIG. 6 is a flowchart of aspects of the system;

FIG. 7 is a flowchart of aspects of the system;

FIG. 8 is a flowchart of aspects of the system;

FIG. 9 is an example of the appearance and functionality of aspects of the system;

FIG. 10 is an example of the appearance and functionality of aspects of the system;

FIG. 11 is an example of the appearance and functionality of aspects of the system;

FIG. 12 is an example of the appearance and functionality of aspects of the system;

FIG. 13 is an example of the appearance and functionality of aspects of the system;

FIG. 14 is an example of the appearance and functionality of aspects of the system;

FIG. 15 is an example of the appearance and functionality of aspects of the system;

FIG. 16 is an example of the appearance and functionality of aspects of the system;

FIG. 17 is a display and output of aspects of the system; and,

FIG. 18 is a display and output of aspects of the system.

### DETAILED DESCRIPTION OF THE INVENTION

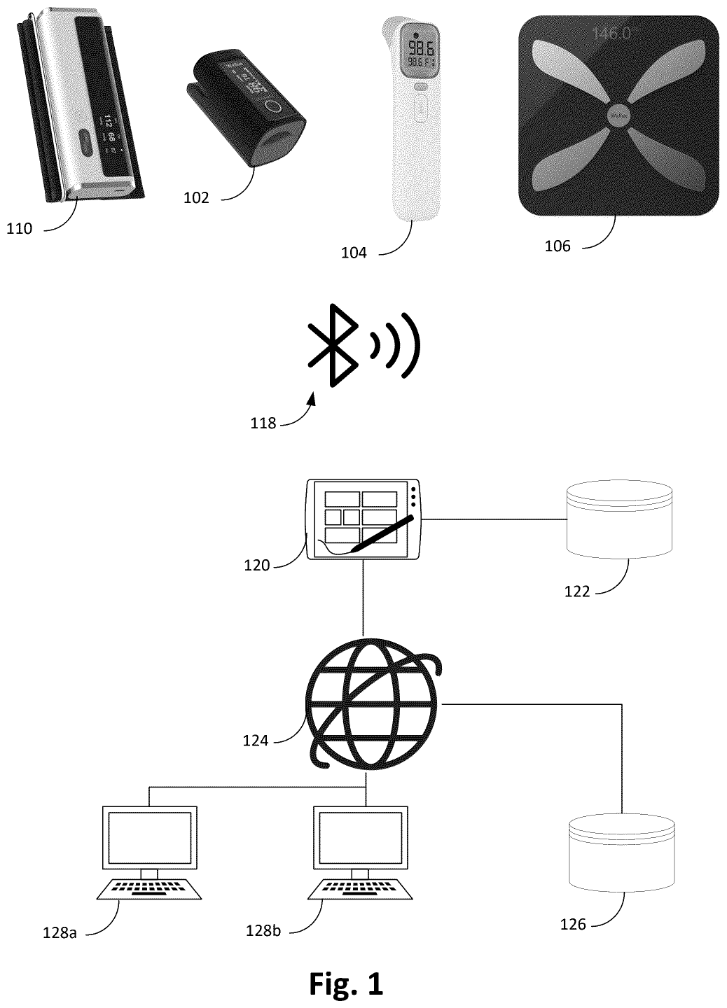

With reference to the drawings, the invention will now be described in more detail. Referring to FIG. 1, the aspects of the system are shown including data capture devices for blood pressure 110, heart rate (e.g., pulse) 102, temperature (thermometer) 104 and weight (scale) 106. The blood pressure capture device can display systolic and diastolic results using an inflatable cuff in communication with a blood pressure computer system. The pulse oximeter can provide SpO₂%, heart beats per minute and power index. The thermometer can be infrared and provide temperature in Celsius or Fahrenheit. The scale can display results in pounds or kilograms.

These devices can be used by the health care provider or the patient. These devices can be in communications with a computer system 120 through wireless communications that can include Bluetooth 118, Wi-Fi, near field RF, cellular, and IR, which can be an independent network or included in an existing network. Local computer system 120 can include a smart phone, laptop, table, or other portable computer device. The computer system can use a communications system 124 allowing information to be transmitted and received from remote third-party systems such as 28a through 128b or a local database 122. For example, a patient can use the various hardware and sensors to capture and record health information (e.g., vital signs) that can be stored on the local computer system and transmitted elsewhere. The local computer system or the remote computer systems can receive the data and analyze the data and store the data on a remote database 126 that can be accessed (including with proper permissions) by third parties such as health care providers and payment entities. In one embodiment, the data corrected can be compared to a range of acceptable value.

For example, the patient can use the pulse oximeter to measure O₂ level which generally should be above 95%. When the O₂ falls below 95%, it can be an indication that health is needed. When SpO₂ falls below 95%, the local or remote computer system can contact a health care provider. Further, the acceptable range can value from individual to individual. For a certain patient may routinely have a SpO₂ at 90% which can be considered normal for that patient. In this case, the system can transmit the SpO₂ to a health care provider, the health care provider can determine that this is a “normal” reading for that patient and the local or remote computer system can reduce the acceptable range of SpO₂ from 95% to 90%. Therefore, a feedback loop is created allowing the system to adjust normal ranges for the particular patient.

Further, when the system determines that a value is out of a normal range, the system can request that the patient capture health care data more frequently. For example, if the SpO₂ is being measured once a day and one reading falls below 95%, the system can request that the patient capture SpO₂ in a shorter period of time. If a patient is shivering, shaking, or has some other movement during the capture of SpO₂, it is possible that the reading will be artificially low. Therefore, the system may request a subsequent reading in a reduced time period to determine if the low reading is a trend or a one-off reading.

The system can also correlate one reading with another. For example, if the SpO₂ is low and the pulse rate rises during the reading, one possible indication is that the patient is in motion which could negatively affect the accuracy of the SpO₂ reading. The system can instruct the patient to remain still and request that the SpO₂ be read again. If the information (e.g., SpO₂) remains lower than the acceptable range, the system can call a health care provider, call for medical transportation (e.g., ambulance), instruct the patient to contact a healthcare provider, take some other action, and any combination thereof.

The application can be used for data capture, data display, data aggregation, and any combination. Different embodiments of the application can provide for one or more of these functionalities exclusive (e.g., only data capture) or can provide for multiple functionality (e.g., data recording and display). The user of the application can have data access rights associated so that the user can access captured data, the associated patient information, EMR information associated with the patient from an EMR, and any combination.

The devices can access wireless access points of a dedicated network or non-dedicated network. The information transmitted from the device to the computer system can be encrypted in transit. The computer system can be in direct communication with the device or through a local area network, wide area network, or global area network.

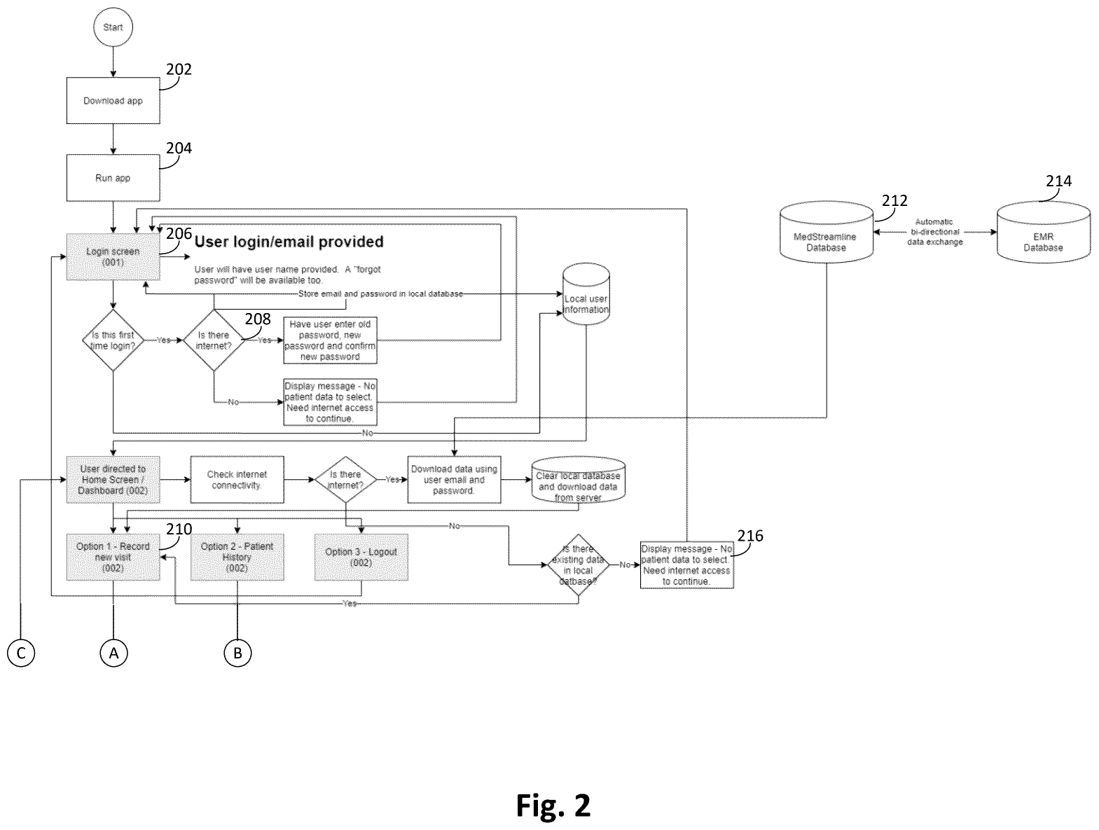

The computer device can include computer readable instructions that have the functionalities shown in FIGS. 2 through 19. Referring to FIG. 2, a local computer system can have a set of computer readable instructions (e.g., app) downloaded at 202 that can be executed at 204. A log in process 206 can be implemented that can include security features such as biometrics. In the event that the local computer device has a local, wide, or global communications network connection at 208, a handshake can be performed so that information can be transmitted to and from the local computer system from a remote computer system. The remote computer systems can include health care systems, EMR, payment system, auditing systems, and any combination thereof. A determination can be made at 210 if this is a new patient or if the patient is preexisting. If there is a preexisting patient, historical information can be present from the local database or remote database such as 212 and 214. In one embodiment, if there is no record of the patient locally or remotely, the system can terminate at 216 or wait for a network connection to proceed.

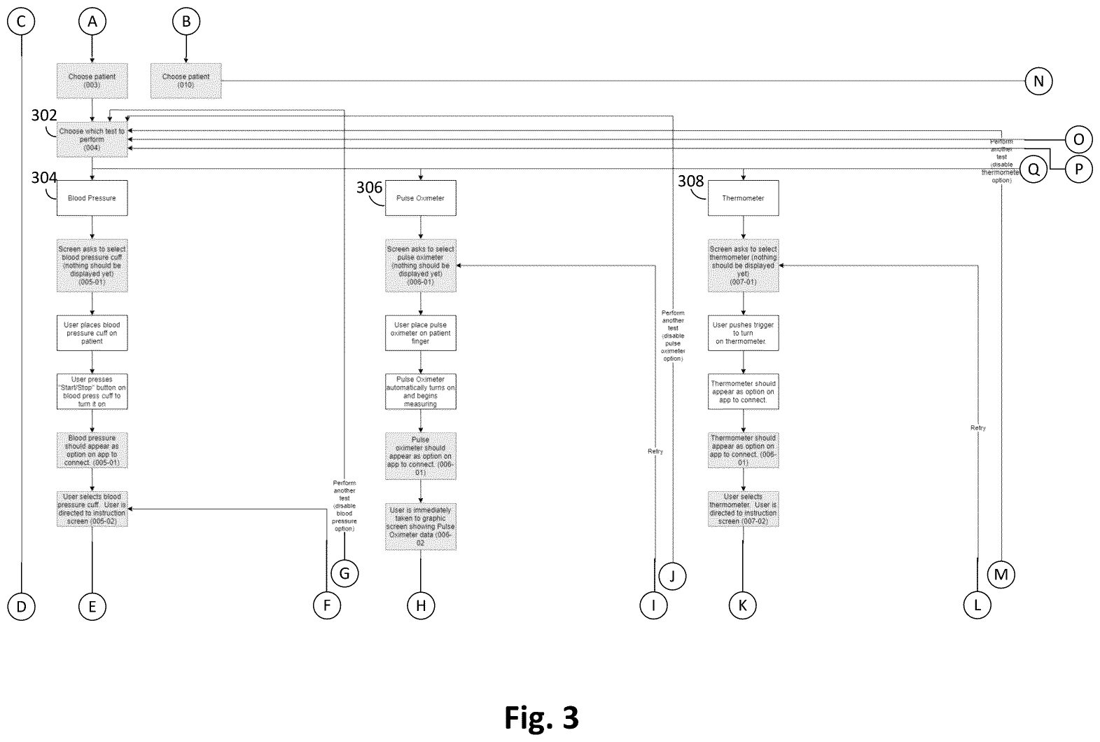

Referring to FIG. 3, the computer readable instructions, locally or remotely (e.g., local or SaaS), can retrieve the patient information, including historical information, and await instructions as to which health care data is to be collected at 302. In one embodiment, blood pressure is measured at 304. In this example, the computer readable instructions can perform a handshake with the blood pressure reader and identify the make and model of the blood pressure reader. The system can retrieve medical device information and provide for on screen use instructions. The information that is received from the computer system can determine the sequence of images and instructions that the computer system displaces. For example, the instructions can request that the patient void the patient's bladder and rest for a certain period of time (e.g., five minutes). If information is received from the medical device within the period requested for rest, it can indicate that the patient is noncompliant, and the computer system can verify that the patient complied with each instruction. When a blood pressure cuff is applied, fit can be determined when inflated so that if the cuff is not properly placed on the patient, the computer system can instruct the patient to do so. The computer instructions can also request that a second reading be taken within a few minutes of the first reading so that the value scan be compared for consistency as well as the account for variations in the readings.

The computer system can also correlate reading from different medical devices to determine if there is an explanation for a value that is out of an acceptable range. In one example, the computer system can request that the patient verify that the patient rested for a certain period of time prior to having the blood pressure taken and if not, the blood pressure being higher than normal can be noted that it may be the result of the patient not resting prior to the reading being taken. The data received from one medical device can also assist with determining if a value is out of range. For example, if the ambient temperature is higher than normal, the patient temperature and even blood pressure can be higher than normal. The ambient temperature can be read from a sensor such as the thermometer prior to its application to the patient.

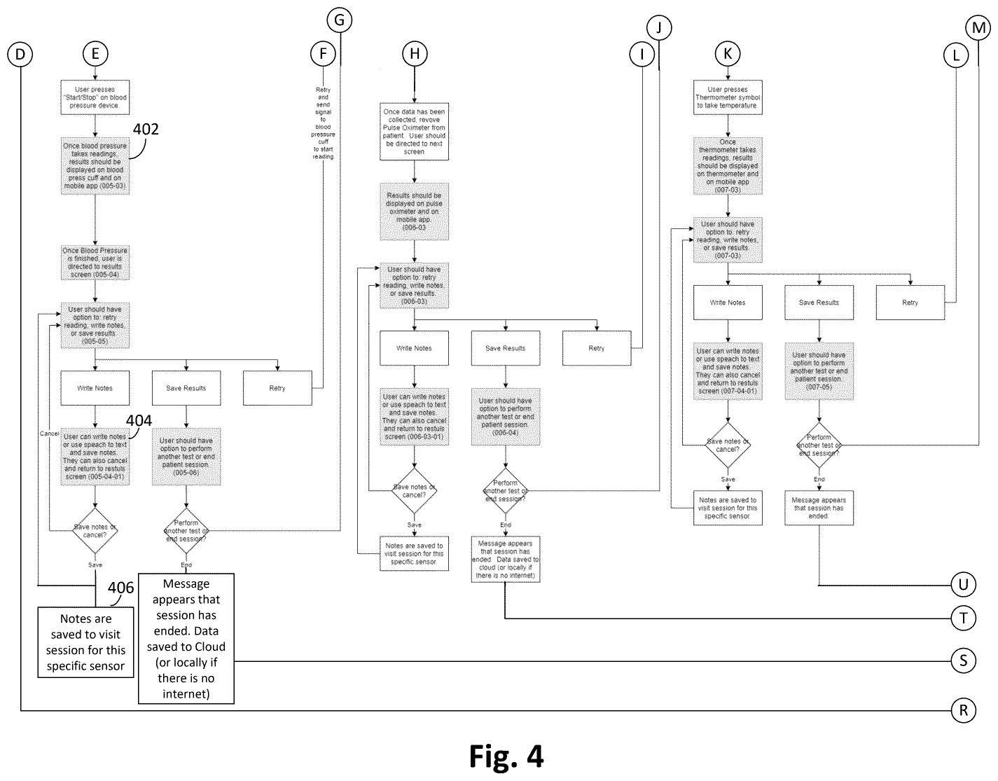

Referring to FIG. 4, reading from the medical device (e.g., blood pressure device can be displayed on the device and the computer system at 402. The patient or other individual can enter notes into the computer system and the notes can be associated with the readings. The notes can be inputted at text, graphic, audio and any combination thereof at 404. The information can be saved in one or more databases in communications with the medical device, at 406.

The data, once collected, can be included in a location database 122 (FIG. 1) or can be transmitted to a remote database (e.g., cloud storage) 126. The captured data can be accessed through a local computer device 120 or by remote computer devices 128a and 128b (e.g., patient computer system and health care provider computer system. In one example, the patient can use one or more devices to collect health care information. The data and time that the data is collected can be stored with the health care data. This information can be stored on local database 122 or remote 126. The health care provider can access the data and review the data prior to or during telecommunications visit with the patient so that the patient does not need to travel to the location of the health care provider. The health care provider can also instruct the patient to collect health care data during the telecommunications visit (e.g., inline visit) so that the health care provider can be provided health information in real time.

The collected health care data can be stored in a EMR that can be accessed by the health care provider or other healthcare providers so that the data need not be captured multiple times for each health care provider.

Referring to FIG. 2, the SpO₂ can be measured by a pulse oximeter at 306 and the computer system can provide instructions for its use. In one embodiment, the computer system can have a first medical device (e.g., blood pressure sensor) and a second medical device (e.g., pulse oximeter) so that when the second medical device detects a reading that is outside an acceptable range, the computer system can retrieve the reading from the first medical device and make a determination that the second medical device's reading may be due to a non-compliant patient. For example, the SpO₂ may be low and the blood pressure may be high indicating that patient is in motion which can negatively affect the readings. The computer device can include sensors such as an accelerator that can determine if the patient is in motion when the readings are received. In one embodiment the local compute device is a smart phone which can detect motion.

The computer readable instruction on a local computer device can be in the formation of a Software as a Service (SaaS) that can function on any number of local devices that include a tablet (e.g., Android, Windows, or iOS operating systems), smartphone, personal computer, laptop, and the like. The data from the data capture devices can be transmitted securely to a remote computer system including a health care professional computer system. The patient data can be reviewed remotely using a patient monitoring dashboard. The dashboard can graphically display the information of the patient in text, charts, graphs, and the like. The date, time, and other such data taken for each medical device can be included in the received data so that a temporal data record can be developed for that patient. This information can be helpful as in one study, it was reported that oxygen saturation increases over the course of the night in mountaineers at high altitudes. The local computer device can include altimeters and location sensors allowing the health care data to be collected and associated with a date, time, and location. Therefore, if the readings of the medical device are outside normal ranges, the date, time, and location can be reviewed and used for determination of security signaling warnings, as needed.

For example, if the patient is taking a reading in the morning and is at a high altitude, the oxygen saturation can be increased as well as systolic blood pressure and diastolic blood pressure.

The medical device can be a thermometer at 308 and can be in communication with the location computer device. The computer system can provide instructional information to the patient on the use of the temperature sensor. The instructions can be device specific as the computer system can receive information from the medical device. The temperature can be captured, notes added, and the information stored. The patient can be prompted to take another reading of the temperature and the session can be ended.

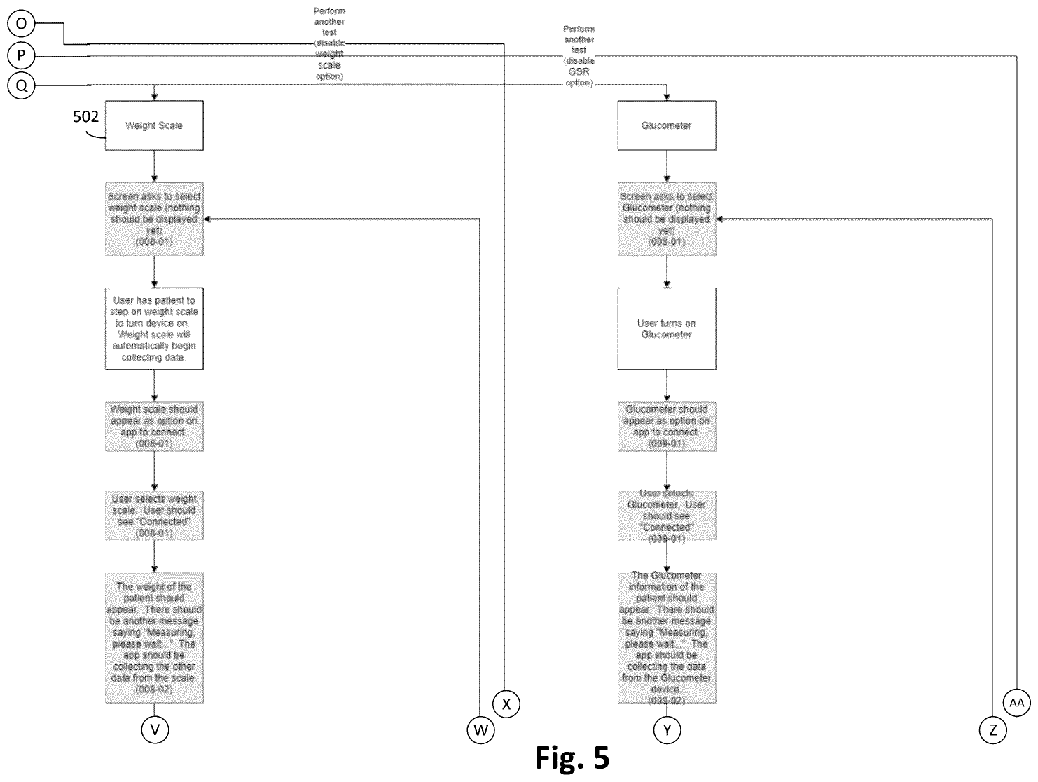

Referring to FIG. 5, the patient can select the weight scale or can be prompted to capture weight data at 502. The computer system can calibrate the scale and instruct the patient as to its use. The instructions for the scale and other medical device can be altered according to the prior medical information received as well as the data history, date, time, and location. For example, the patient may have a normal range where the blood pressure is maintained. However, the blood pressure device can detect either once or over time a rise in the blood pressure. The scale can, over the same period of time, determine that there is an increase in weight. As increase in weight can be a factor of an increase in blood pressure, the computer systems ability to match these vital signs with date, time, and location can assist the medical professional in rendering health care services. The computer system can generate output as shown in FIG. 20 showing the weight, systolic, and diastolic blood pressure over time and in relation with a loss of weight.

Referring to FIG. 5, a glucometer can be in communications with the computer device and can be used for recording blood glucose levels. These levels can be captured at a specific location, date, and time and therefore, correlated to other vital sign readings. High blood glucose levels can cause widespread damage to tissues and organs, including those that play a key role in maintaining healthy blood pressure. For example, damage to the blood vessels and kidneys can cause blood pressure to rise. Therefore, the computer system can identify that the increase in blood pressure can be associated with high blood glucose levels and present this information to the patient. In many instances, the inability of the health care provider to correlate the blood glucose with blood pressure can result in a missed diagnosis. The human body has limited options for raising blood pressure. The human body relies on the same hormones that are activated when blood glucose is low, including catecholamine, or adrenaline, hormones. Catecholamines tighten up our blood vessels, making it harder for the heart to pump the blood through our arteries, which raises blood pressure. Therefore, the computer system in this invention correlates that these vital signs provide for a comprehensive vital sign data capture that can be displayed over time, date, and location and displayed with every other reading.

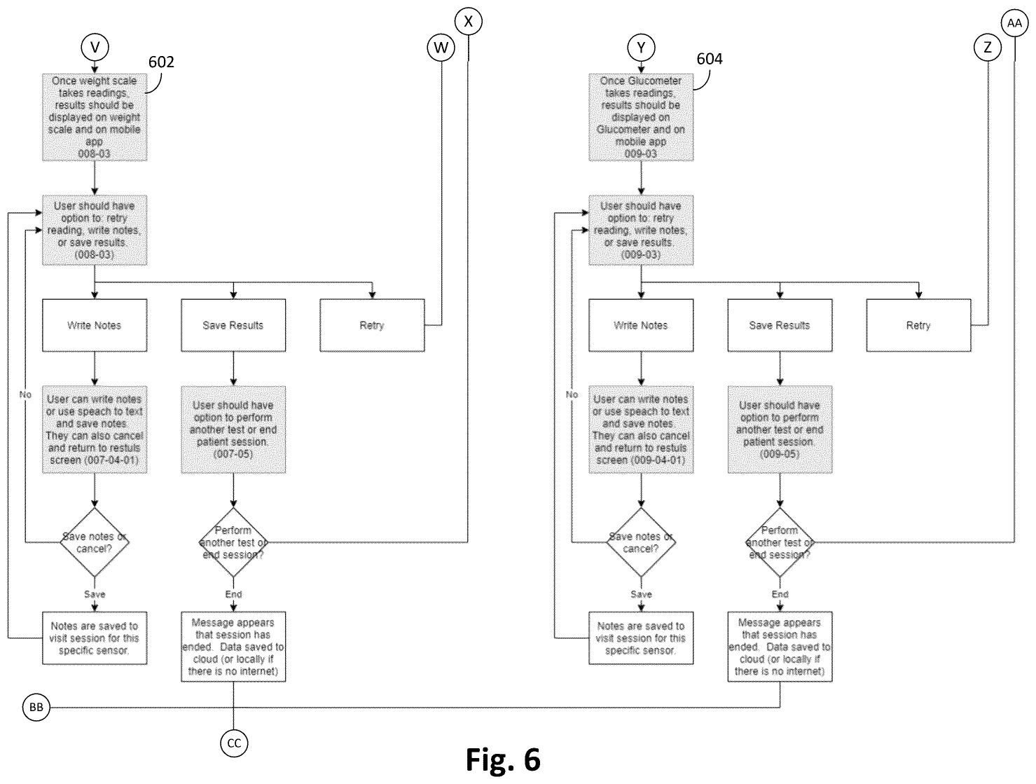

Referring to FIG. 6, the scale at 602 and the glucometer at 604 can be displayed to the user, displayed on the computer device, local and remote, and stored on one or more databases. Notes can be added as well as the date, time, and location of the data capture. If internet connectivity is lacking, the information can be stored locally, or the process can be suspended until communications are established.

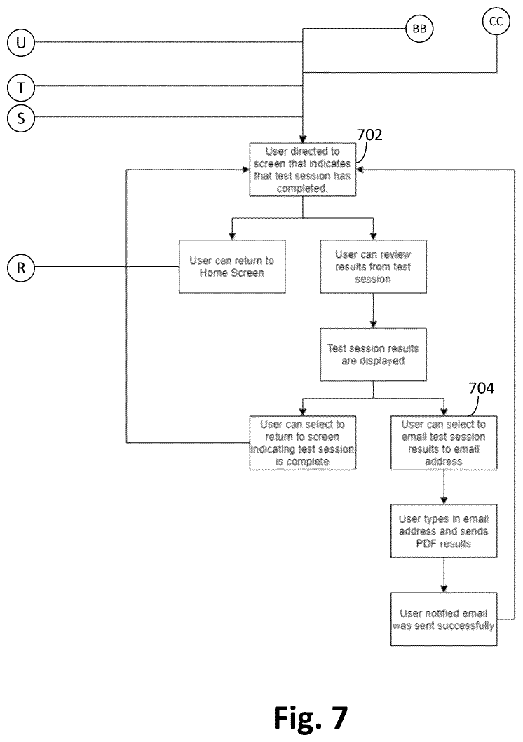

Referring FIG. 7, the patient can be informed that all data has been collected at 702 or some other termination event occurs such as the computer system informs the patient to immediately call emergency health care services. The results can be transmitted to a third party automatically or at the request of the patient, such as emailing the results at 704.

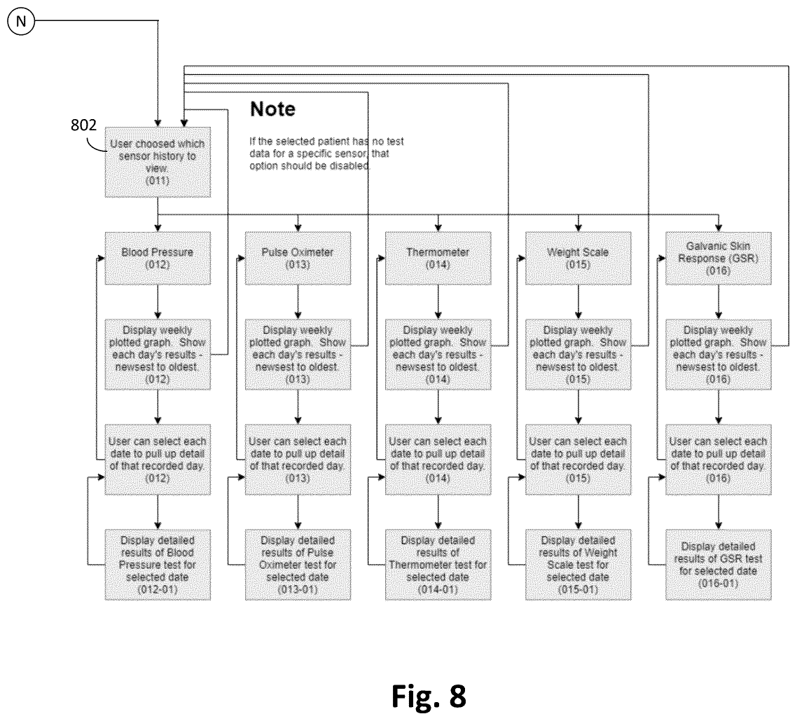

Referring to FIG. 8, the data that is captured is saved so that the history of the data can be viewed at the patient's request at 802. The data can include text or graphic format and can include any one of blood pressure, pulse oximeter, thermometer, weight, galvanic skin response, and any combination. As to galvanic skin response, it can be an indication of the state of the patient and can include an indication as to the increase in eccrine sweat gland activity when the patient is saddened, threatened, joyful, or other emotion, and this information can be overlayed and correlated with the prior vital sign readings. Other sensors that can be included are EEG sensors.

The patient can record data from capture devices using a software application and can be used to access a cloud-based application or locally stored application. The patient or health care provider can access the application and thereby access the patient health information. In one embodiment, some users can access multiple patients' information (e.g., health care providers) while other users can only access their personal data.

In the event that the application is not connected to a remote database or a EMR system, the captured data can be stored locally on a computer device and transmitted to a remote system (remote database and EMR) once an internet connection is detected. When the data is uploaded to a remote system, the data stored locally can be deleted to improve security in that the data is not present at the local computer device.

The data to be collected can be recorded and can be taken from a group of devices that can collect data including blood pressure, pulse, temperature, weight, and glucose (e.g., the level of glucose in the blood). The user can use the device which will then upload information to a local or remote system that is associated with a patient record. By associating the information with the patient record, the system can integrate the collected data with a EMR that has also has an associated patient record. Due to this system's ability to transmit data to an EMR, the present system does not rely upon a specific EMR nor does it need a specific EMR for its functionality. When the data is captured, the results can be displayed on the device and the application. Once captured, the user (patient or health care provider) can retry the data collection, provide notes, and save the results. The notes can be provided with speech to text through the application. In the event that the local computer device does not have a connection to a database, the application can display that the session has ended, and data can be saved locally.

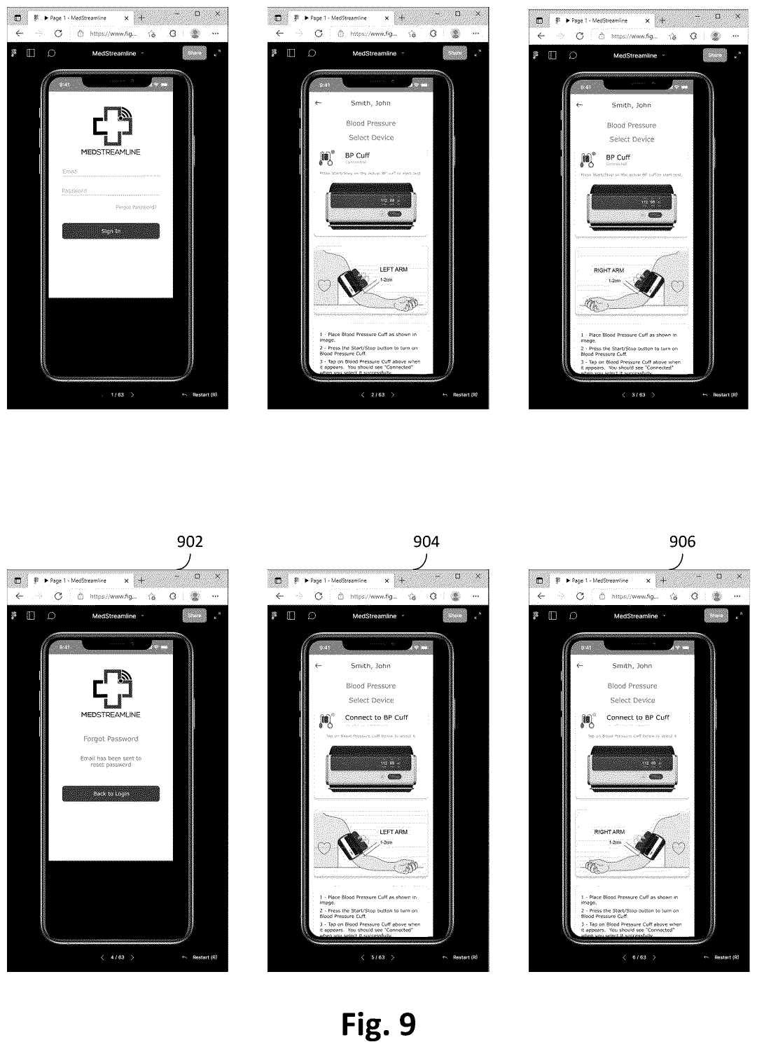

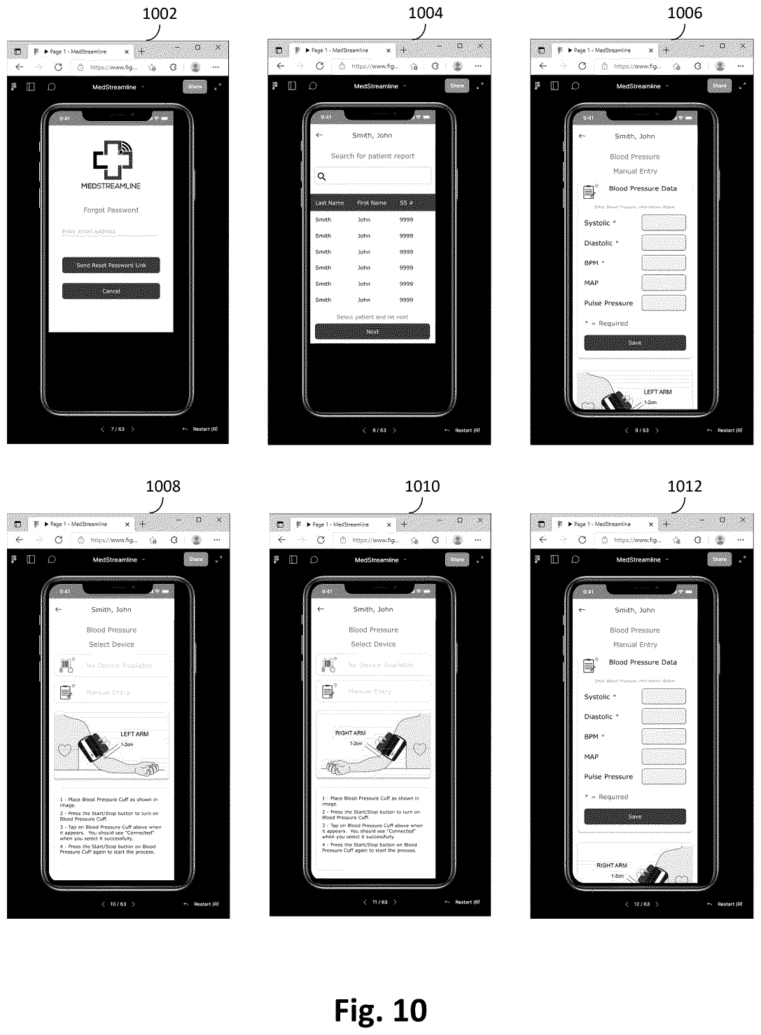

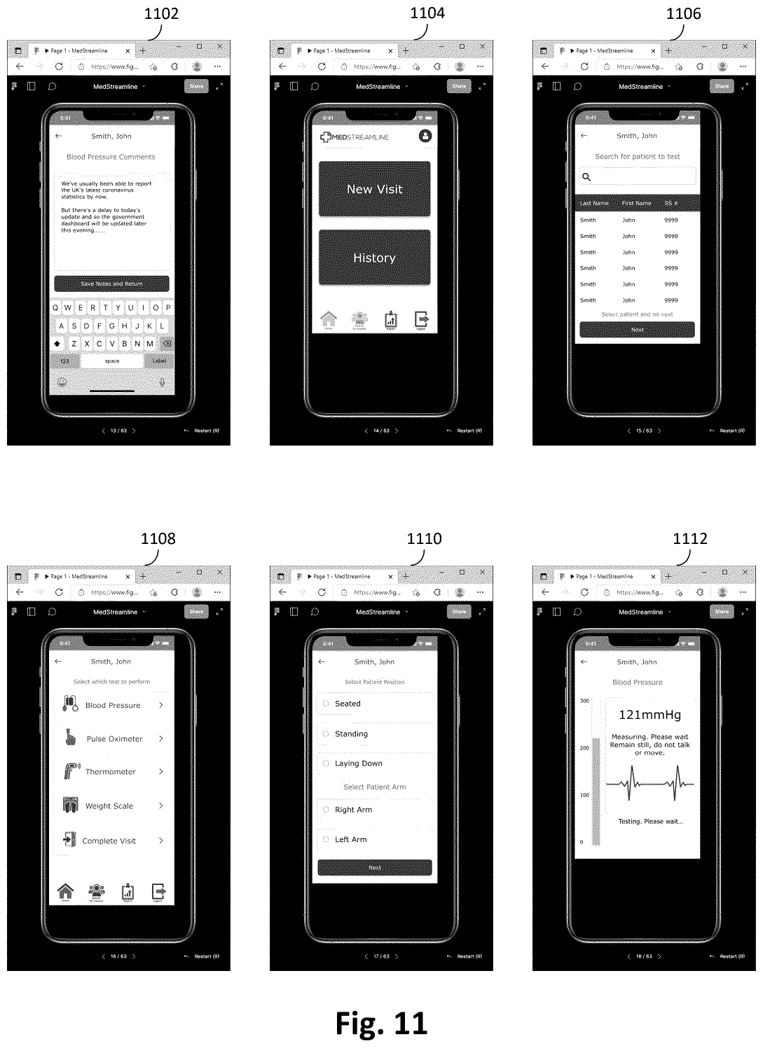

Referring to FIG. 9 and by way of example, the computer system can direct the patient on the user of a blood pressure monitor, as shown. The patient can provide identification information at 902. The patient can receive instructions to connect the blood pressure cuff at 904 and 906. Referring to FIG. 10, the patient can reset the identification information and access information at 1002. The patient can be identified and associated with historical data at 1004. The results can be manually entered at 1006. In the event that the reading should be captured a second or subsequent time, the computer system can provide instructions at 1008 and 1010. The results can be manually entered at 1012 if needed. Referring to FIG. 11, notes can be added at 1102. The patient can elect to enter new data or review historical data at 1104 and the patient selected at 1106. The data to be collected can be selected at 1108 or can be selected automatically when the computer system detects the presence of a medical device. If the computer system detects the presence of more than one medical device, the computer system requests that the patient use a first medical device and then a second medical device according to a predetermined priority. If the computer system detects the presence of more than one medical device, the computer system displays the detected device selection and requests that the patient elect a device to first use. If the device is used and data is gathered, the device can be removed from the available options of medical device until each device detected is used by the patient. In one embodiment, the orientation of the patient can be entered at 1110. This information can be used in the interpretation of the medical information. For example, it's typical that blood pressure falls slightly when someone stands. However, if the individual stands for a longer period of time, blood pressure can increase. Therefore, the computer system can determine the time between when the orientation of the patient is entered and the period when the medical information is captured. The results can be displayed at 1112.

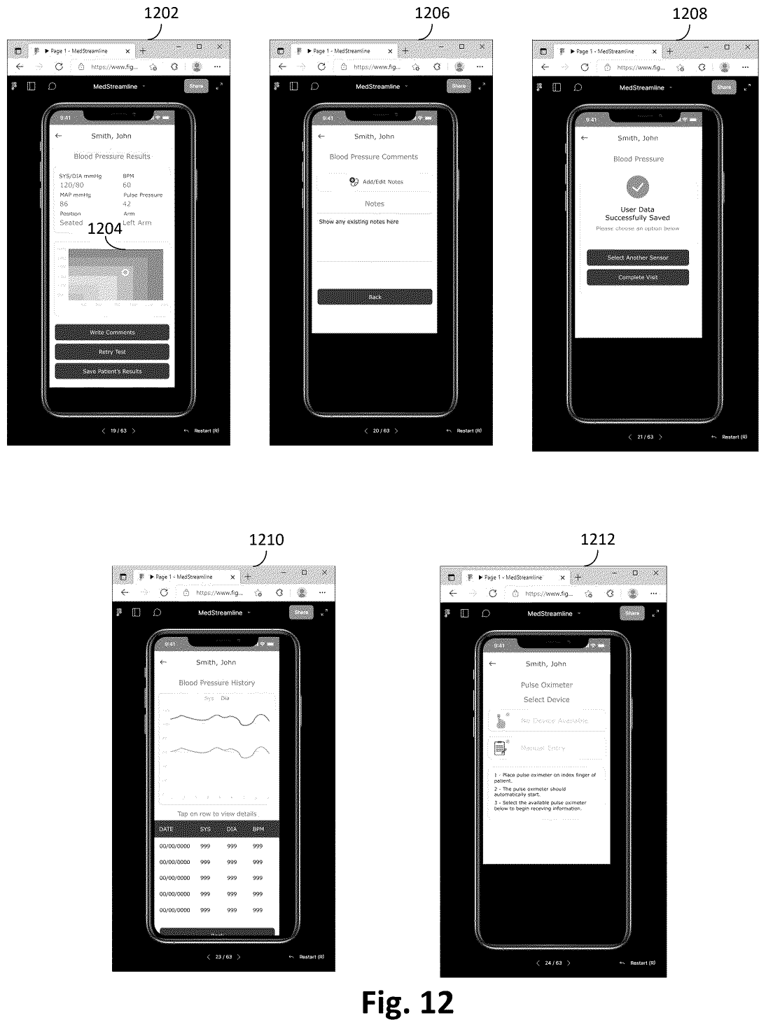

Referring to FIG. 12, the information can be displayed in graphical form at 1202. The acceptable ranges 1204 can have a default value and then can be modified according to a series of data captures and the input from a medical professional. The ranges can provide an indication as to the acceptable values of the medical device measurements as well as to determine what instructions or communications, if any, the computer system can make. In one example, when the heart rate is high and the patient is experiencing a heart attack, a higher risk of mortality occurs, especially at rate higher than 80 beats per minute. Also, during a heart attack, blood pressure can drop. Therefore, in one scenario, high pulse rate with low blood pressure can trigger a warning to the patient or automatic communication to a health care provider. When blood pressure is measured, notes can be provided at 1204. When the data is successfully saved, an indication of the success saving can be shown, as 1208. The history of the data (e.g., blood pressure) can be displayed at 1210. The next medical device can be used at 1212.

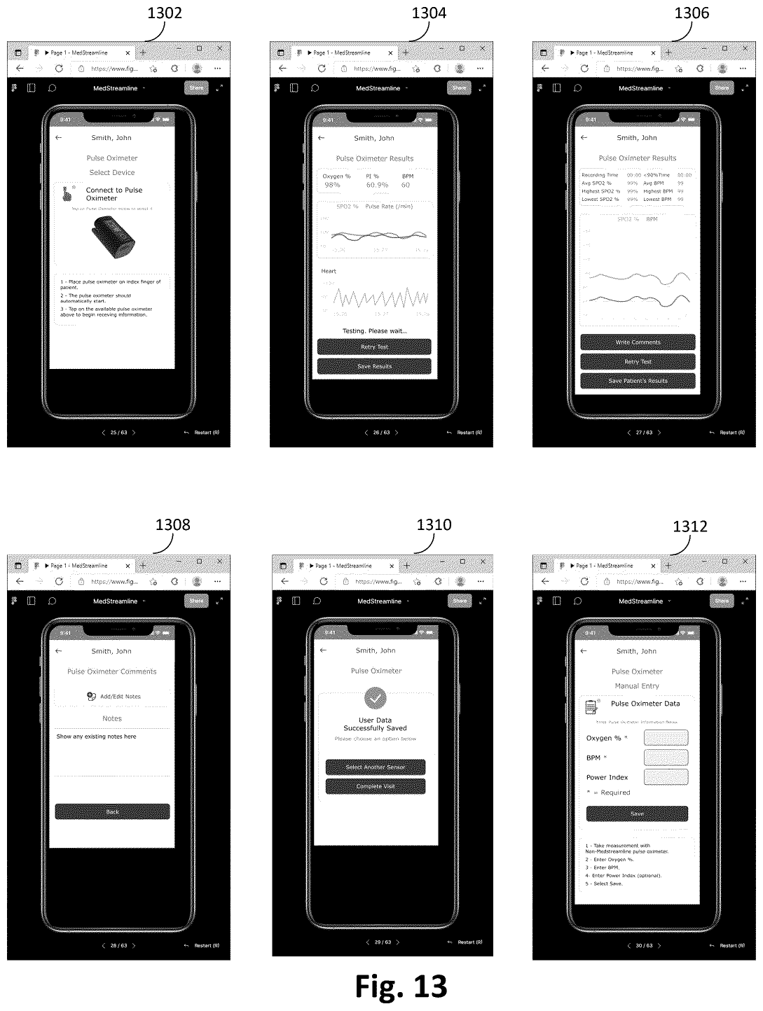

Referring to FIG. 13 at 1302, the second medical device can be shown to the patient so that the patient can identify the next medical device to use. The data can be captured at 1304, which demonstrates testing is occurring and the results are then displayed at the local computer device, at 1306, and transmitted to be stored. Notes can be added at 1308. An indication that the data has been successfully saved can be shown at 1310. The results can be manually entered at 1312 if needed.

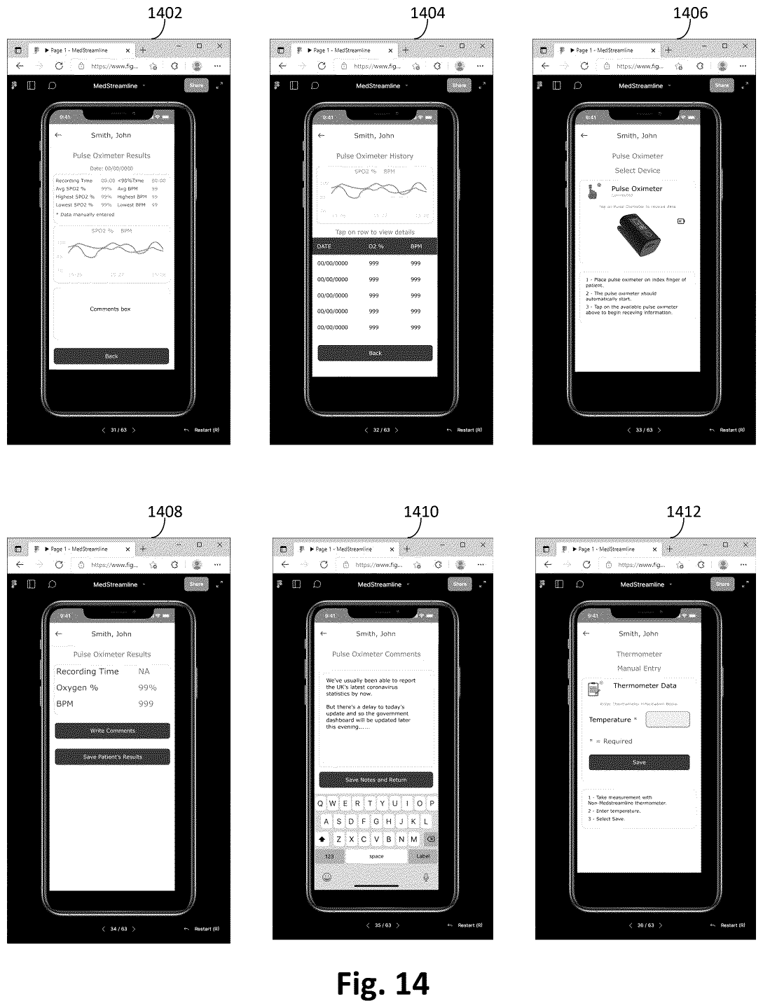

Referring to FIG. 14, results can be displayed at text or graphically at 1402. The results can be displayed at 1404 and the instruction for the use of the medical device can be shown as 1406. The results can be displayed with an indication that the data was manually entered and was not received directly from the medical device as shown in 1402. The patient, or other individual, can be prompted to enter notes or save the information at 1408 and 1410.

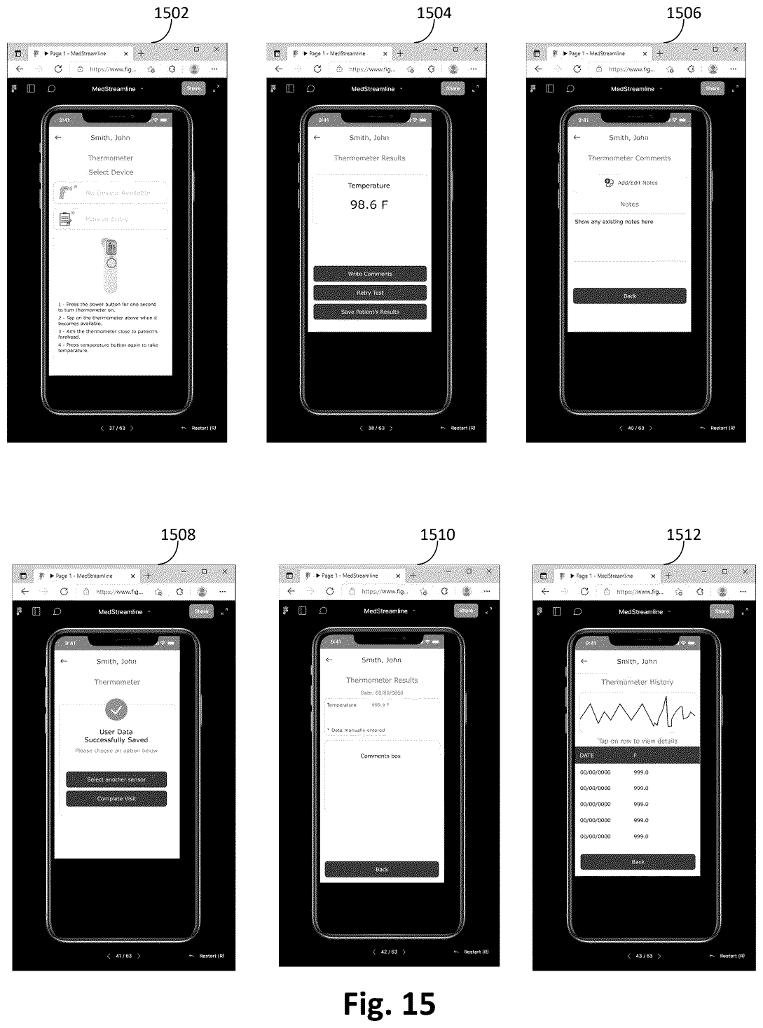

For the thermometer, the information can be manually entered at 1412. Referring to FIG. 15, instruction for the thermometer can be shown at 1502 when the thermometer can communicate with the computer device. The results can be displayed at 1504. Notes can be added at 1506. The data can be shown to have been saved at 1508 and shown to have been manually entered at 1510. The history of the data can be shown at 1512.

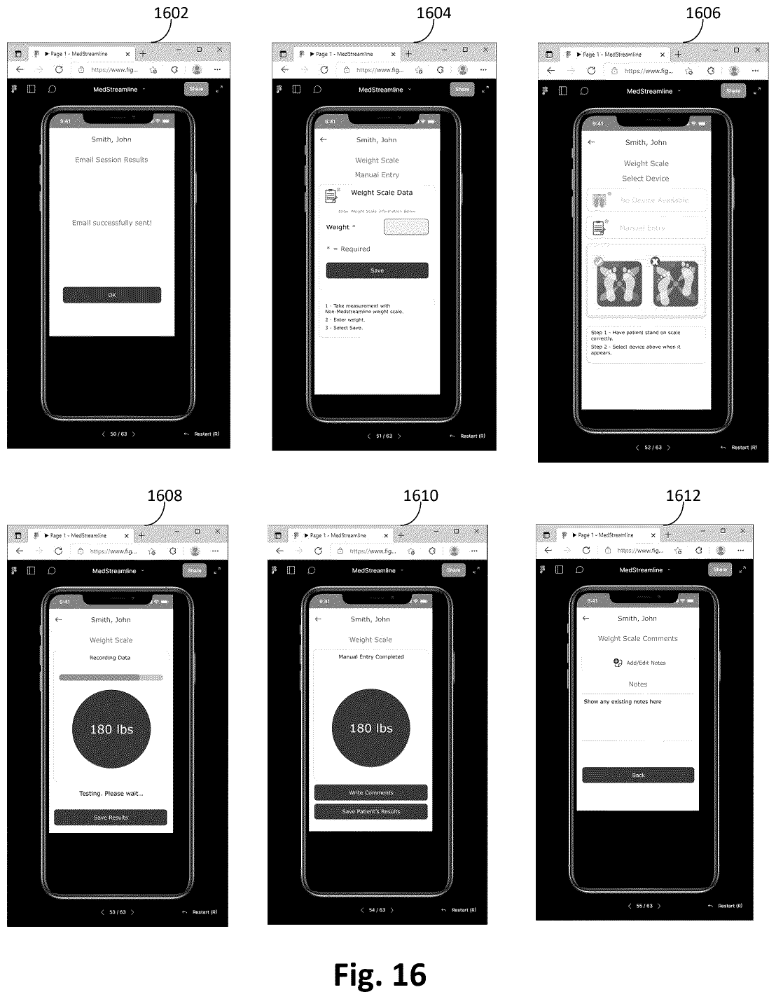

Referring to FIG. 16, the data can be sent to a third party at 1602 and confirmation shown on the computer device. For weights, the data can be entered manually at 1604. The patient can be provided instructions at 1606 that can be device dependent according to the information that is received from the medical device. The medical device can provide type, makes, model, series, and information allowing the computer system to determine the proper instructions and information to send and receive from the medical device. For example, the computer system can display an image of the device at 1608 and show proper placement of the patient in relation with the device. The results can be shown during the gathering/capturing process at 1608 and the results at 1610. Notes can be entered at 1612 and associated with the patient, date, time, and location.

In one embodiment, the user can be directed to know that a data capture session has ended and provide the options to review the test results, exit the application, restart the session, retake a specific test, or preform the data collection again, have the results sent by electronic message (including a link to remotely stored data) or send the results in another format including PDF. Electronic communications can be provided to the user that inform the user of the state if the data such as “saved,” “uploaded,” “integrated into EMR,” “upload failed,” or other status of the data transmission and storage may be completed.

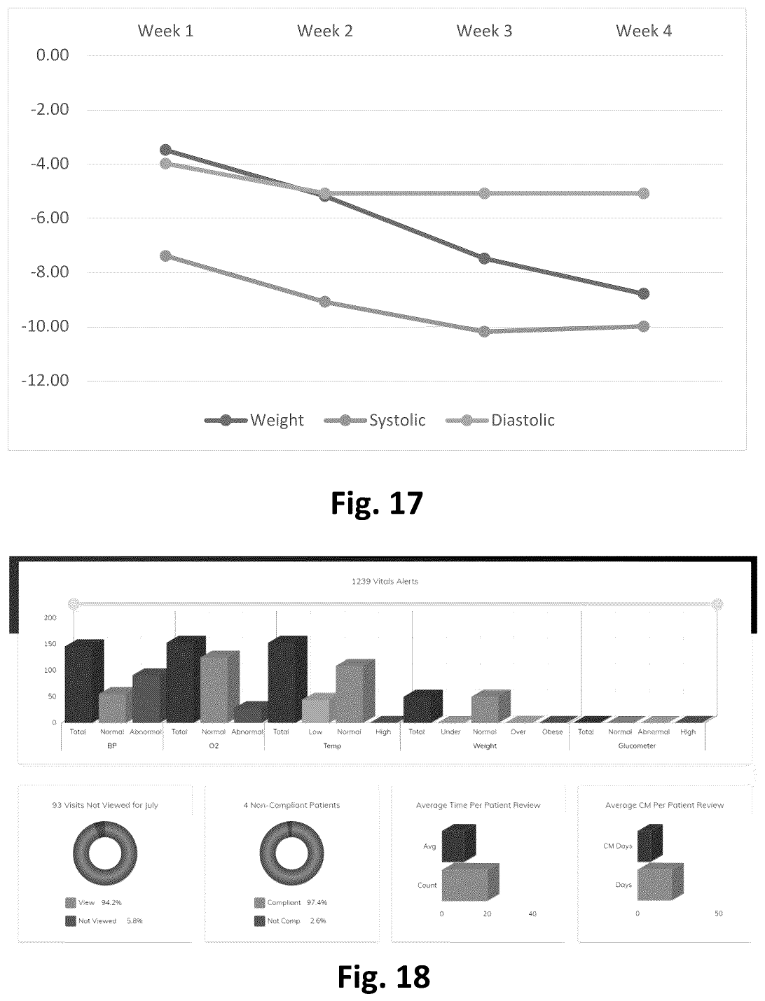

The data collected for each device (e.g., sensor) can be stored in and retrieved as an aggregation of the data session. The data collected (e.g., testing results) can be displayed in text or graphical form as shown in FIG. 17 to provide the user with an indication of the change in the measured health information over time. For example, the blood pressure can be viewed in association with the data and time captured to determine if blood pressure is increasing over time, decreasing over time, changes with the time of day or other temporal relationships. The computer system can indicate if the device is able to transmit data to the application (e.g., connected) or if the application cannot detect that the device is present or operational (e.g., not connected).

Referring to FIG. 18, the computer system can also gather information, aggregate the information, and display the information for use by an entity such as a health care provider, assisted living facility, and the like. The computer system can determine if health care information has not been collected and can generate a warning that the health care information should be collected.

The system can also assist with monitoring the health care services provided. The computer system can receive health information from a patient and inform a health care provider that the information is ready for review. Therefore, the health care provider has access to the health information from many patients, even when at a disparate location and decentralized, and can review the information efficiently using the computer system. The computer system can track what patient information has been viewed by the health care provider as well as what patient information has not been viewed. In the event that health care information has not been viewed, the computer system can provide a warning or send communications to the health care provider (e.g., email or text) that can remind the health care provider to review the information.

The health care information itself can be aggregated and displayed as a total of the health care information gathered, the number of abnormal readings, the number of normal readings, the number of records viewed, the average time that the health care provider viewed the record. Therefore, this information can be used to determine efficiency, processes, procedures, and the like for a patient, health care provider, and even a facility. For example, if one facility using the computer system shows the average time per patient review being less than a second facility, the first facility may be experiencing staffing shortages.

In the event that there is not health care information gathered and the information shows non-compliance, the computer system can display the listing of non-compliant patients so that the health care information can be gathered to prompt the patient into compliance.

A summary report can be generated showing the health care session that can include the vital signs captured, recording time, review time, and the like.

It is understood that the above descriptions and illustrations are intended to be illustrative and not restrictive. It is to be understood that changes and variations may be made without departing from the spirit or scope of the following claims. Other embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventor did not consider such subject matter to be part of the disclosed inventive subject matter. The images and the flow charts contained herein are those that are used by one skilled in the art to show a sequence of operations computer readable code must perform. The images and the flow charts are used by one skilled in the art to explain and understand computer readable instructions and the functionality contained therein.

What is claimed is:

1. A computerized information management system comprising:
a local computer system having a computer readable medium;
a first medical device in communications with the local computer system adapted to capture a first set of medical data from a patient;
a second medical device in communication with the local computer system adapted to capture a second set of medical data from the patient;
a remote computer system in communication with the local computer system; and
a set of local computer readable instructions adapted to, receive a first medical device information from the first medical device, display a first use instruction according to the first medical device information, receive a first patient medical information from the first medical device, display the first patient medical information on the local computer system, display a first warning according to the first patient medical information being outside a first acceptable range, receive a second medical device information from the second medical device, display a second use instruction according to the second medical device information, receive a second patient medical information from the second medical device, display the second patient medical information on the local computer system, display a second warning according to the second patient medical information being outside a second acceptable range, display a correlation information according to a comparison of the first patient medical information and the second patient medical information, and transmit the first patient medical information and the second patient medical information to the remote computer system.

2. The information collection system of claim 1, further comprising:
a patient record stored on a database and associated with a patient representing historical medical information; and
wherein the set of local computer readable instructions are adapted to modify the patient record according to information taken from a group consisting of the first medical device information, first patient medical information, second medical device information, second patient medical information, date, time, location, and any combination thereof.

3. The information collection system of claim 1, further comprising:
a patient record stored on a database and associated with a patient representing historical medical information; and
wherein the set of local computer readable instructions are adapted to retrieve the patient record and display a warning if the first medical device information is outside a historical range.

4. The information collection system of claim 1, further comprising:
a patient record stored on an electronic medical record system; and
wherein the set of local computer readable instructions are adapted to amend the patient record with information taken from the group consisting of the first patient medical information, the first medical device information, the second patient medical information, the second medical device information, date, time, location, and any combination thereof.

5. The information collection system of claim 1, wherein the set of local computer readable instructions are adapted to display the correlation information according to a comparison of the first patient medical information and the second patient medical information wherein the first patient medical information affects the second patient medical information.

6. The information collection system of claim 1, wherein the set of local computer readable instructions are adapted to receive the first patient medical information from the first medical device by manual entry.

7. The information collection system of claim 1, wherein the set of local computer readable instructions are adapted to request that the patient actuate the first medical device to receive a third medical device information from the first medical device.

8. The information collection system of claim 5, wherein the set of local computer readable instructions are adapted to request that the patient actuate the first medical device to receive a third medical device information from the first medical device according to a comparison of the first patient medical information within the first acceptable range.

9. The information collection system of claim 5, wherein the set of local computer readable instructions are adapted to request that the patient actuate the second medical device to receive a third medical device information from the second medical device according to a comparison of the first patient medical information within the first acceptable range.

10. The information collection system of claim 1, wherein the set of local computer readable instructions are adapted to transmit a health message to a health care provider according to the first patient medical information being outside the first acceptable range and an approval to transmit from the patient.

11. An computerized information management system comprising:
a local computer system having a computer readable medium and in communication with an electronic medical record system;
an initial patient record stored on a database in communication with the local computer system;
a first medical device in communication with the local computer system adapted to capture a first set of medical data from a patient;
a second medical device in communication with the local computer system adapted to capture a second set of medical data from the patient;
a remote computer system in communication with the local computer system; and
a set of local computer readable instructions adapted to receive a first medical device information from the first medical device, display a first use instruction according to the first medical device information, receive a first patient medical information from the first medical device, display the first patient medical information on the local computer system, receive a second medical device information from the second medical device, display a second use instruction according to the second medical device information, receive a second patient medical information from the second medical device, display the second patient medical information on the local computer system, and create an amended patient record according to the initial patient record and information taken from a group consisting of the first medical device information, first patient medical information, the second medical device information, the second patient medical information, date, time, location, and any combination thereof.

12. The information collection system of claim 11, wherein the set of local computer readable instructions are adapted to retrieve the initial patient record from the electronic medical record system, amend the initial patient record and store the amended patient record on the database wherein the database is included in the electronic medical record system.

13. The information collection system of claim 11, wherein the set of local computer readable instructions are adapted to transmit the amended patient record to the remote computer system.

14. The information collection system of claim 11, wherein the first medical device is taken from the group consisting of a blood pressure sensor, pulse sensor, thermometer, weight scale, and galvanic skin sensor.

15. The information collection system of claim 11, wherein the set of local computer readable instructions are adapted to instruct the patient to actuate the first medical device after a period of time from receiving the first patient medical information and receiving a third patient medical information from the first medical device.

16. The information collection system of claim 15, wherein the set of local computer readable instructions are adapted to display a correlation information according to a comparison of the first patient medical information and the third patient medical information.

17. A computerized information management system comprising:
a local computer system having a computer readable medium and in communications with an electronic medical record system;
an initial patient history stored on a database in communication with the local computer system wherein the initial patient history is associated with a patient;
a first medical device in communication with the local computer system adapted to capture a first set of medical data from the patient;
a second medical device in communication with the local computer system adapted to capture a second set of medical data from the patient; and
a set of local computer readable instructions adapted to receive a first medical device information from the first medical device, display a first use instruction according to the first medical device information, receive a first patient medical information from the first medical device, receive a second medical device information from the second medical device, display a second use instruction according to the second medical device information, receive a second patient medical information from the second medical device, append the initial patient history according to information taken from the group consisting of the first patient medical information, the first medical device information, the second patient medical information, the second medical device information, date, time, location, and any combination thereof.

18. The information collection system of claim 17, wherein the database is included in the electronic medical records system.

19. The information collection system of claim 17, wherein the set of local computer readable instructions are adapted to append the initial patient history by associating information taken from the group consisting of the first patient medical information, the first medical device information, the second patient medical information, the second medical device information, date, time, location, and any combination thereof, with the initial patient history.

20. The information collection system of claim 19, wherein the set of local computer readable instructions are adapted to transmit a health message to a health care provider according to the first patient medical information being outside a first acceptable range.
